(19) United States
(12) Patent Application Publication
(10) Pub. No.: US 2026/0111633 A1
(43) Pub. Date: Apr. 23, 2026

(54) AUTOREGRESSIVE LARGE LANGUAGE MODEL BASED GENERATION OF CHEMICAL STRUCTURE DESCRIPTIONS

(71) Applicant: Sciome, LLC, Research Triangle Park, NC (US)

(72) Inventors: Christopher NORMAN, Fagersta (SE); Brian HOWARD, Raleigh, NC (US); Ruchir SHAH, Durham, NC (US)

(21) Appl. No.: 19/364,741

(22) Filed: Oct. 21, 2025

Related U.S. Application Data
(60) Provisional application No. 63/709,907, filed on Oct. 21, 2024.

(51) Int. Cl. G06F 30/27; G16C 20/70

(52) U.S. Cl. CPC G06F 30/27; G16C 20/70

(57) ABSTRACT

The subject matter described herein includes a method for generating chemical structure descriptions. Is some aspects, the method includes training a machine learning (ML) model using a training set comprising molecules described using a line notation for describing chemical structures, the line notation having a vocabulary of tokens. The method further includes using the trained ML model to generate chemical structures described using the line notation for describing chemical structures. The chemical structures may be generated with or without providing a prompt to the ML model.

### CROSS-REFERENCE TO RELATED APPLICATION

This application claims priority to U.S. Provisional Patent Application No. 63/709,907, filed Oct. 21, 2024, entitled “AUTOREGRESSIVE LARGE LANGUAGE MODEL BASED GENERATION OF CHEMICAL STRUCTURE DESCRIPTIONS”, which is assigned to the assignee hereof and is expressly incorporated herein by reference in its entirety.

### BACKGROUND

Generative artificial intelligence (AI) refers to the use of a trained machine learning (ML) model to create something in response to an input prompt. One type of generative AI model is an autoregressive large language model (LLM). A large language model (LLM) is a computational model notable for its ability to achieve general-purpose language generation and other natural language processing tasks such as classification. LLMs acquire these abilities by learning statistical relationships from vast amounts of text during a computationally intensive self-supervised and semi-supervised training process. LLMs can be used for text generation, a form of generative AI, by taking an input text and repeatedly predicting the next token or word.

LLMs are artificial neural networks that utilize the transformer architecture, invented in 2017. A transformer is a deep learning architecture based on a multi-head attention mechanism. Text is converted to numerical representations called tokens, and each token is converted into a vector via look up from a word embedding table. At each layer, each token is then contextualized within the scope of the context window (a finite set of previously seen and/or generated tokens) via a parallel multi-head attention mechanism allowing the signal for key tokens to be amplified and less important tokens to be diminished.

The term autoregressive indicates that the output variable depends on its own previously predicted values; thus, the model can be expressed in the form of a recurrence relation. Autoregressive LLMs form the basis for all large language models such as GPT-3, GPT-4, Claude and similar models. Autoregressive language models follow the following basic algorithm.

First, initialize the generated list of tokens with the input prompt given by the user, broken down into tokens. Then, until the model has generated a stopping token or the maximum number of output tokens has been reached, do the following: for each token T in the vocabulary, use the language model to predict the likelihood that T will be the next token, given the list of tokens that has already been generated; then randomly choose between the most likely token and a less likely token according to a preset temperature, and add the selected token to the generated list of tokens. Once the model has generated a stopping token or the maximum number of output tokens has been reached, the generated list of tokens is converted into a string and provided to the user as the output.

### SUMMARY

The subject matter described herein includes a method for generating chemical structure descriptions. In some aspects, the method includes training a machine learning (ML) model using a training set comprising molecules described using a line notation for describing chemical structures, the line notation having a vocabulary of tokens. In some aspects, the ML model comprises an autoregressive large language model (LLM). The method further includes using the trained ML model to generate chemical structures described using the line notation for describing chemical structures. The chemical structures may be generated with or without providing a prompt to the ML model.

According to one aspect, the subject matter described herein includes methods for generating chemical structure descriptions. In some aspects, the method includes training an ML model using a training set comprising molecules described using a line notation for describing chemical structures, the line notation having a vocabulary of tokens. The method further includes using the trained ML model to generate chemical structures described using the line notation for describing chemical structures.

According to another aspect, the subject matter described herein includes an apparatus for generating chemical structure descriptions. In some aspects, the apparatus includes a memory and at least one processor communicatively coupled to the memory. At least one processor is configured to train a machine learning (ML) model using a training set comprising molecules described using a line notation for describing chemical structures, the line notation having a vocabulary of tokens. At least one processor is further configured to generate, using the trained ML model, chemical structures described using the line notation for describing chemical structures.

The subject matter described herein for autoregressive LLM-based generation of chemical structure descriptions may be implemented in hardware, software, firmware, or any combination thereof. As such, the terms “function” or “module” as used herein refer to hardware, software, and/or firmware for implementing the feature being described. In one exemplary implementation, the subject matter described herein may be implemented using a computer readable medium having stored thereon executable instructions that when executed by the processor of a computer control the computer to perform steps. Exemplary computer readable media suitable for implementing the subject matter described herein include disk memory devices, chip memory devices, programmable logic devices, application specific integrated circuits, and other non-transitory storage media. In one implementation, the computer readable medium may include a memory accessible by a processor of a computer or other like device. The memory may include instructions executable by the processor for implementing any of the methods described herein. In addition, a computer readable medium that implements the subject matter described herein may be located on a single device or computing platform or may be distributed across multiple physical devices and/or computing platforms.

### BRIEF DESCRIPTION OF THE DRAWINGS

The accompanying drawings are presented to aid in the description of various aspects of the disclosure and are provided solely for illustration of the aspects and not limitation thereof.

FIG. 1A shows the chemical structure for a simple chemical compound, and FIG. 1B shows the mapping between a SMILES notation string for that compound and the structural components of that compound.

FIG. 2 is a system diagram illustrating an example system for autoregressive large language model (LLM) based generation of chemical structure descriptions, according to aspects of the disclosure.

FIGS. 3-5 are flowcharts showing methods for autoregressive LLM-based generation of chemical structure descriptions, according to aspects of the disclosure.

### DETAILED DESCRIPTION

Presented herein are techniques for autoregressive LLM-based generation of chemical structure descriptions. Unlike conventional autoregressive LLMs, which are trained on natural language to generate natural language output, the autoregressive LLMs in the present disclosure are trained on chemical structure descriptions to generate chemical structure descriptions. In some aspects, the autoregressive LLMs are trained chemical structures as described using the simplified molecular-input line-entry system (SMILES) notation.

Aspects of the disclosure are provided in the following description and related drawings directed to various examples provided for illustration purposes. Alternate aspects may be devised without departing from the scope of the disclosure. Additionally, well-known elements of the disclosure will not be described in detail or will be omitted so as not to obscure the relevant details of the disclosure.

The words “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspects of the disclosure” does not require that all aspects of the disclosure include the discussed feature, advantage or mode of operation.

Those of skill in the art will appreciate that the information and signals described below may be represented using any of a variety of different technologies and techniques. For example, data, instructions, commands, information, signals, bits, symbols, and chips that may be referenced throughout the description below may be represented by voltages, currents, electromagnetic waves, magnetic fields or particles, optical fields or particles, or any combination thereof, depending in part on the particular application, in part on the desired design, in part on the corresponding technology, etc.

Further, many aspects are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, the sequence(s) of actions described herein can be considered to be embodied entirely within any form of non-transitory computer-readable storage medium having stored therein a corresponding set of computer instructions that, upon execution, would cause or instruct an associated processor of a device to perform the functionality described herein. Thus, the various aspects of the disclosure may be embodied in a number of different forms, all of which have been contemplated to be within the scope of the disclosed subject matter. In addition, for each of the aspects described herein, the corresponding form of any such aspects may be described herein as, for example, “logic configured to” perform the described action.

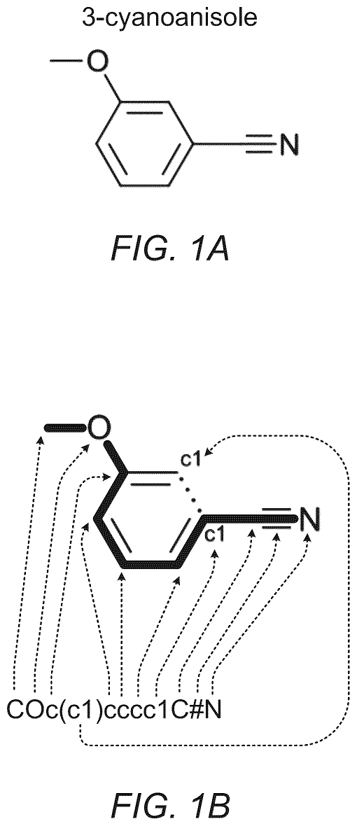

FIG. 1A shows the chemical structure for 3-cyanoanisole, and FIG. 1B shows the mapping between a SMILES notation string for 3-cyanoanisole and the structural components of 3-cyanoanisole. SMILES notation describes ring structures, such as the 6-carbon ring within 3-cyanoanisole, by breaking each ring at an arbitrary point to make an acyclic structure and adding numerical ring closure labels to show connectivity between non-adjacent atoms. FIG. 1B shows the location of the break with a dotted line, and the use of the closure label “1”. As shown in FIG. 1B, breaking the carbon ring creates a string of atoms that form a main backbone (indicated in FIG. 1B by a thicker line), from which branches may extend.

The SMILES description of the chemical structure illustrated in FIG. 1B is “COc(c1)cccc1C #N”, where “C” represents a carbon atom that is not part of a ring, “c” represents a carbon atom that is part of a ring, “O” represents an oxygen atom, “N” represents a nitrogen atom, a set of parentheses indicates the contents of a branch off of the main backbone, and “#” represents a triple bond. An equal sign “=” represents a double bond, and single bonds are presumed unless otherwise indicated, so no symbol is needed to indicate a single bond. Other examples of SMILES strings are shown in Table 1, below:

TABLE 1
Example SMILES strings

| Molecule | SMILES formula |
|---|---|
| Dinitrogen | N#N |
| Vanillin | O═Cc1ccc(O)c(OC)c1 |
| Melatonin | CC(═O)NCCC1═CNc2c1cc(OC)cc2 |
| Flavopereirin | CCc(c1)ccc2[n+]1ccc3c2[nH]c4c3cccc4 |
| Nicotine | CN1CCC[C@H]1c2cccnc2 |
| Oenanthotoxin | CCC[C@@H](O)CC\C═C\C═C\C#CC#C\C═C\CO |
| Glucose | OC[C@@H](O1)[C@@H](O)[C@H](O)[C@@H](O)[C@H](O)1 |
| Bergenin | OC[C@@H](O1)[C@@H](O)[C@H](O)[C@@H]2[C@@H]1c3c |
| | (O)c(OC)c(O)cc3C(═O)O2 |
| Thiamine | OCCc1c(C)[n+](cs1)Cc2cnc(C)nc2N |
| Cephalostatin-1 | CC(C)(O1)C[C@@H](O)[C@@]1(O2)[C@@H](C)[C@@H]3CC═ |
| | C4[C@]3(C2)C(═O)C[C@H]5[C@H]4CC[C@@H](C6)[C@]5 |
| | (C)Cc(n7)c6nc(C[C@@]89(C))c7C[C@@H]8CC[C@@H]%10[C |
| | @@H]9C[C@@H](O)[C@@]%11(C)C %10═C[C@H](O |
| | %12)[C@]%11(O)[C@H](C)[C@]%12(O% 13)[C@H](O)C[C@@]%13(C)CO |

Rather than training an autoregressive LLM on natural language inputs, from which the model “learns” the rules of proper syntax and grammar of the language (e.g., English), the autoregressive LLM of the present disclosure is trained on SMILES strings, from which the model “learns” the underlying principles that govern chemical structures.

Not only is the autoregressive LLM trained on SMILES strings rather than natural language, the process that is used to generate a string of tokens differs from conventional processes as well. After using the language model to predict the likelihood that T will be the next token, rather than choosing the most likely token, the next token to be added to the output string is chosen randomly according to the predicted probabilities.

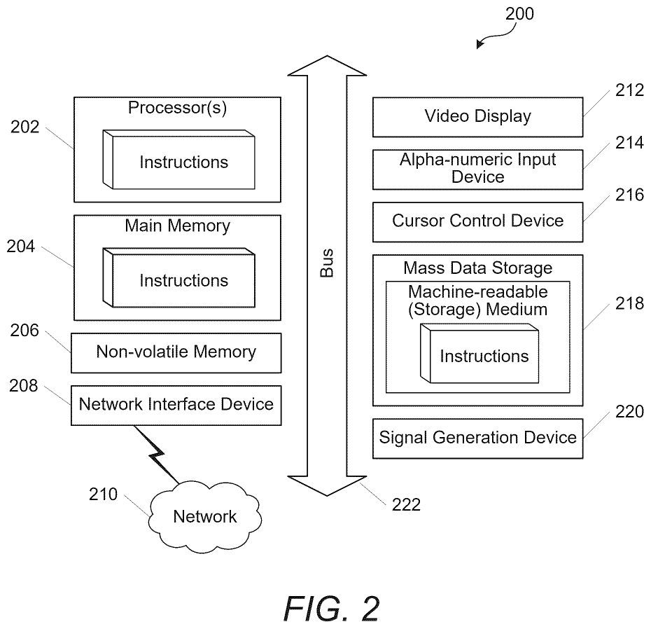

FIG. 2 is a system diagram illustrating an example system 200 for detection and elimination of duplicate literature from a literature set, according to aspects of the disclosure. FIG. 2 shows a diagrammatic representation of a machine in the example form of a computer system within which a set of instructions for causing the machine to perform any one or more of the methodologies discussed herein may be executed.

In the example of FIG. 2, the computer system 200 includes at least one processor 202, main memory 204, non-volatile memory 206, and an interface device 208 for connecting to a network 210. System 200 may include a video display 212, an alpha-numeric input device 214, such as a keyboard or touch screen, a cursor control device 216, such as a mouse, trackpad, touchpad, or touch screen, and non-volatile mass data storage 218, such as a hard disk drive, solid state drive, etc. System 200 may include a signal generation device 220, such as a speaker or microphone. Memory 204 is coupled to processor 202 by, for example, a bus 222.

In some aspects, system 200 can train a machine learning (ML) model using a training set comprising molecules described using a line notation for describing chemical structures, the line notation having a vocabulary of tokens. In some aspects, the system 200 can use an ML model that has been trained elsewhere and provided to the system 200. In some aspects, the system 200 can use the trained ML model to generate chemical structures described using the line notation for describing chemical structures. In some aspects, the ML model is stored in main memory 204, non-volatile memory 206, mass data storage 218, or any combination thereof. In some aspects, the ML model is located on the network 210 and accessed by the system 200 via the network interface device 208.

Various common components (e.g., cache memory) are omitted for illustrative simplicity. The system 200 is intended to illustrate a hardware device on which any of the components depicted in figures or described in this specification can be implemented. The system 200 can be of any applicable known or convenient type. The components of the system 200 can be coupled together via a bus or through some other known or convenient device.

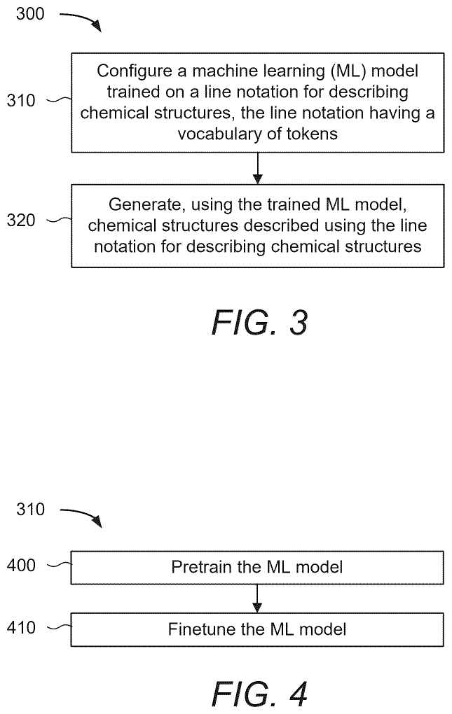

FIG. 3 is a flowchart illustrating a method 300 for autoregressive LLM-based generation of chemical structure descriptions, according to aspects of the disclosure. In the example shown in FIG. 3, the method 300 includes, at block 310, training an ML model using a training set comprising molecules described using a line notation for describing chemical structures, the line notation having a vocabulary of tokens. In some aspects, the ML model comprises an autoregressive large language model. In some aspects, the line notation for describing chemical structures is the simplified molecular-input line-entry system (SMILES). In the example shown in FIG. 3, the method 300 further includes, at block 320, using the trained ML model to generate chemical structures described using the line notation for describing chemical structures.

FIG. 4 is a flow chart showing the operation of block 310 (training the ML model) in more detail, according to an aspect of the disclosure. In some aspects, training the ML model comprises pretraining the ML model using a first plurality of general molecule descriptions and finetuning the ML model using a second plurality of target molecule descriptions.

In the example shown in FIG. 4, training the ML model comprises pretraining the ML model (block 400). In some aspects, a model will first be pretrained on a general dataset. During pretraining, the model will learn the general rules of the language, such as grammar, syntax and word choice. Where the model is pretrained on SMILES strings, for example, the model will learn the general rules of grammar and syntax for SMILES strings. In some cases, such pretrained models are too general to be useful to end-users and are therefore commonly finetuned for specific use cases. Thus, in some aspects, the model may be pretrained on a publicly available library of compounds and their SMILES descriptions; for example, the PubChem database includes descriptions of 87 million compounds.

In the example shown in FIG. 4, training the ML model further comprises finetuning the ML model (block 410). In some aspects, the model may then be finetuned using a targeted subset of compounds; for example, thousands of previous drug candidates may be used. In the above example, the model uses the publicly available database to learn the SMILES syntax, which results in what is referred to as a “pre-trained” model, then uses the previous drug candidates to finetune the selection probabilities so that the model generates descriptions of chemical compounds that more closely resemble the kinds of compounds that are of interest, which produces a model known as a “checkpoint.”

In some aspects, low-rank adaptation (LoRA) is used as a method for fine-tuning the machine learning (ML) model. LoRA is a technique that adapts a pre-trained language model to a specific task by introducing additional trainable rank-decomposition matrices into each layer of the neural network, while keeping the original pre-trained weights frozen. By doing so, LoRA significantly reduces the number of trainable parameters required during fine-tuning. This reduction not only decreases the computational resources needed but also accelerates the training process, making it faster and more cost-effective than full fine-tuning of all model parameters. Since a LoRA adapter only consists of low-rank matrices, this approach also requires only a fraction of the storage requirements of non-LoRA finetuning. In some aspects, finetuning may be performed without LoRA, but LoRA makes the process faster and allows training using less expensive GPU hardware.

For example, after pre-training the autoregressive LLM on a general dataset of molecules represented using SMILES notation, LoRA adapters can be employed to fine-tune the model for generating chemical structures with specific properties or functionalities. A LoRA adapter corresponding to a particular class of compounds (e.g., antiviral agents) can be trained by adjusting only the low-rank matrices introduced by LoRA, leaving the rest of the model unchanged. This approach allows the model to capture the nuances and patterns specific to the target class without overwriting the general chemical knowledge acquired during pre-training. Moreover, LoRA adapters are modular and largely orthogonal, meaning multiple adapters trained on different properties or compound classes can be combined or swapped freely. The model can consequently be adapted for various tasks by simply loading different LoRA adapters as needed.

The use of LoRA adapters improves the performance of the LLM in generating chemical structures in several ways. Firstly, because LoRA fine-tuning focuses on a smaller subset of parameters, it requires less data and computation to achieve convergence, resulting in quicker turnaround times compared to full fine-tuning. Secondly, by fine-tuning only the low-rank matrices, the model retains the general chemical knowledge from pre-training while specializing in the target domain, leading to more accurate and relevant outputs. For instance, a model fine-tuned with a LoRA adapter on a dataset of kinase inhibitors may generate chemical structures that not only adhere to the general rules of chemistry but also possess characteristics common to known kinase inhibitors. Furthermore, LoRA adapters can be combined freely, allowing for the generation of compounds that satisfy multiple criteria simultaneously without having to finetune for each specific combination of criteria. Thus, LoRA allows separating some of the finetuned models into reuseable components (the LoRA adapters) that can be quickly swapped in and out based on what kind of compound the user wants to generate. In some aspects, LoRA adapters can be combined with static finetuning.

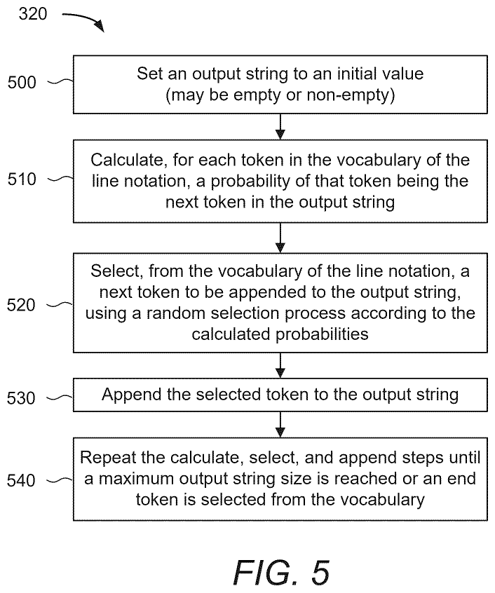

FIG. 5 is a flow chart showing the operation of block 320 (using the trained ML model to generate chemical structures) in more detail, according to an aspect of the disclosure. In the example shown in FIG. 5, generating the chemical structures using the trained ML model comprises setting an output string to an initial value (block 500). In some aspects, setting the output string to an initial value comprises setting the output string to an empty string—e.g., without using a prompt. In some aspects, setting the output string to the initial value comprises setting the output string to the value of an input prompt.

In the example shown in FIG. 5, generating the chemical structures using the trained ML model further includes: calculating, for each token in the vocabulary of the line notation, a probability of that token being the next token in the output string (block 510); selecting, from the vocabulary of the line notation, a next token to be appended to the output string, using a random selection according to the calculated probabilities (block 520); appending the selected token to the output string (block 530); and repeating the calculating, selecting, and appending steps until a maximum output string size is reached or an end token is selected from the vocabulary (block 540).

The chemical structure or structures so generated may then be presented to the user or provided to another step in a larger process. For example, in some aspects, the generated structures can be sorted and/or filtered according to some metric or metrics. In some aspects, the generated structures can be further analyzed to predict chemical characteristics (e.g., toxicity, efficacy, solubility, etc.) and/or fitness for an intended purpose (e.g., interaction with another target molecule), or other analysis based on the chemical structure.

Thus, the methods disclosed herein have one or more of the following features:

First, the autoregressive LLM is trained on SMILES strings, rather than being trained on natural language texts.

Second, the autoregressive LLM will generate SMILES strings that describe chemical structures, rather than generating natural language.

Third, the next token to be added to the output string is chosen randomly according to the predicted probabilities of the vocabulary terms, rather than selecting the most likely token.

Fourth, in some modes of operation, the chemical structure description string is generated from the starting state of an empty output string, i.e., without an input prompt, which can yield a random output distribution of compounds that is not skewed by a prompt. In other modes of operation, the chemical structure description string is generated from the starting state of an output string that has been initialized to the value of an input prompt.

Fifth, the autoregressive LLM generates new compounds, rather than predicting molecular properties from existing chemical structures.

Sixth, new compounds are generated using an autoregressive LLM, rather than using non-LLM methods such as diffusion models, variational autoencoders, generative adversarial networks, recurrent networks, autoregressive graph-based models and normalizing flows.

Seventh, in some aspects, finetuning is used to adapt models to generate only compounds with or without specific properties, and/or with or without specific structures.

Eighth, in some aspects, LoRA is used to finetune the model.

Ninth, in some aspects, LoRAs generate compounds with multiple properties simultaneously.

Tenth, in some aspects, user have access to an online component gallery of checkpoints and low rank adaptation adapters where users can reuse and optionally share their models with each other.

Eleventh, in some aspects, the model is used with a sampling setting (as opposed to a search setting) in order to estimate the distribution of chemicals in the training data.

Twelfth, in some aspects, the model is used with a search setting and a given temperature to generate compounds with a higher (or lower) proportion of specific properties.

The techniques described herein will generate compounds that resemble the compounds present in the training data that was used to train it. In some aspects, to align the generated compounds with user preferences, it may be necessary to perform multiple rounds of training and finetuning with datasets of different composition.

The following is an example scenario in a client-facing pipeline, according to some aspects of the instant disclosure:
1. A client is provided with (or provided access to) a general-purpose model, the base model, which is pretrained on a large number of compounds (on the order of millions). This model is intended to be trained only once.
2. The client is provided with (or provided access to) an intermediate-step model, the checkpoint, which is finetuned from the base model on a smaller number of targeted compounds (on the order of thousands). These kinds of models can be trained several times. In some aspects, this training can be done in the same way as the pretraining in step 1 but can also be initiated (for example) by the client in a software application.
3. The client trains LoRA adapters based on a smaller number of very specific compounds with targeted modalities (on the order of a few dozens). These kinds of adapters can be trained in a few minutes. This training can be done in the same way as the training in step 1 or step 2, or it can be trained based on data the client copies and pastes directly into a GUI.

The client may then choose a suitable checkpoint (e.g., one that generates drug-like compounds that can be synthesized by a particular pharmaceutical manufacturer), and a combination of LORA adapters (e.g., compounds targeting p53 mutations, compounds that can be metabolized by human flavin-containing monooxygenases, etc.). In some aspects, checkpoints and LoRA adapters can be shared by users in a component gallery, online library, online marketplace, etc.

In some aspects, the model can be controlled to generate a larger proportion of compounds with specific properties (leading to greater purity), or a smaller proportion (leader to greater originality) depending on specific client needs. The former decreases the number of candidates that need to be screened, whereas the latter may be useful to ensure no useful candidates are missed. There are several ways to do this:
Replacing the sampling procedure with a search procedure, and adjusting the temperature to generate compounds with different target scores;
Adjusting the distribution of samples in the training data, or calibrating the finetuning to focus on purity or originality; and
Increasing the weights of specific LoRA adapters to increase purity, or decreasing the weights to increase originality.

All the above approaches can be combined freely with each other, and with simpler approaches like filtering.

In the detailed description above it can be seen that different features are grouped together in examples. This manner of disclosure should not be understood as an intention that the example clauses have more features than are explicitly mentioned in each clause. Rather, the various aspects of the disclosure may include fewer than all features of an individual example clause disclosed. Therefore, the following clauses should hereby be deemed to be incorporated in the description, wherein each clause by itself can stand as a separate example. Although each dependent clause can refer in the clauses to a specific combination with one of the other clauses, the aspect(s) of that dependent clause are not limited to the specific combination. It will be appreciated that other example clauses can also include a combination of the dependent clause aspect(s) with the subject matter of any other dependent clause or independent clause or a combination of any feature with other dependent and independent clauses. The various aspects disclosed herein expressly include these combinations, unless it is explicitly expressed or can be readily inferred that a specific combination is not intended (e.g., contradictory aspects, such as defining an element as both an electrical insulator and an electrical conductor). Furthermore, it is also intended that aspects of a clause can be included in any other independent clause, even if the clause is not directly dependent on the independent clause.

Implementation examples are described in the following numbered clauses:

Clause 1. A method for generating chemical structure descriptions, the method comprising: configuring a machine learning (ML) model trained on a line notation for describing chemical structures, the line notation having a vocabulary of tokens; generating, using the ML model, chemical structures described using the line notation for describing chemical structures.

Clause 2. The method of clause 1, wherein the ML model comprises an autoregressive large language model.

Clause 3. The method of any of clauses 1 to 2, wherein configuring the ML model comprises training the ML model using a training set comprising molecules described using the line notation for describing chemical structures.

Clause 4. The method of any of clauses 1 to 3, wherein configuring the ML model comprises receiving and installing a pre-trained ML model.

Clause 5. The method of any of clauses 1 to 4, wherein the line notation for describing chemical structures is simplified molecular-input line-entry system (SMILES).

Clause 6. The method of any of clauses 1 to 5, wherein training the ML model comprises pretraining the ML model using a first plurality of general molecule descriptions and finetuning the ML model using a second plurality of target molecule descriptions.

Clause 7. The method of clause 6, wherein pretraining the ML model using the first plurality of general molecule descriptions comprises pretraining the ML model using molecule descriptions from a public library of molecule descriptions.

Clause 8. The method of any of clauses 6 to 7, wherein finetuning the ML model using the second plurality of target molecule descriptions comprises finetuning the ML model using molecule descriptions of drug candidates.

Clause 9. The method of clause 8, wherein finetuning the ML model comprises finetuning the ML model using low-rank adaptation (LoRA).

Clause 10. The method of clause 9, wherein finetuning the ML model using LoRA comprises introducing additional trainable rank-decomposition matrices into each layer of the neural network, while keeping the original pre-trained weights frozen.

Clause 11. The method of any of clauses 1 to 10, wherein generating the chemical structures using the trained ML model comprises: setting an output string to an initial value; calculating, for each token in the vocabulary of the line notation, a probability of that token being the next token in the output string; selecting, from the vocabulary of the line notation, a next token to be appended to the output string, using a random selection according to the calculated probabilities; appending the next token to the output string; and repeating the calculating, selecting, and appending steps until a maximum output string size is reached or an end token is selected from the vocabulary.

Clause 12. The method of clause 10, wherein setting the output string to an initial value comprises setting the output string to an empty string.

Clause 13. The method of clause 10, wherein setting the output string to the initial value comprises setting the output string to the value of an input prompt.

Clause 14. An apparatus, comprising: a memory; and at least one processor communicatively coupled to the memory, the at least one processor configured to: configure a machine learning (ML) model trained on a line notation for describing chemical structures, the line notation having a vocabulary of tokens; generate, using the ML model, chemical structures described using the line notation for describing chemical structures.

Clause 15. The apparatus of clause 14, wherein the ML model comprises an autoregressive large language model.

Clause 16. The apparatus of any of clauses 14 to 15, wherein to configure the ML model, the at least one processor is configured to train the ML model using a training set comprising molecules described using the line notation for describing chemical structures.

Clause 17. The apparatus of any of clauses 14 to 16, wherein to configure the ML model, the at least one processor is configured to receive and install a pre-trained ML model.

Clause 18. The apparatus of any of clauses 14 to 17, wherein the line notation for describing chemical structures is simplified molecular-input line-entry system (SMILES).

Clause 19. The apparatus of any of clauses 14 to 18, wherein to train the ML model, the at least one processor configured to pretrain the ML model using a first plurality of general molecule descriptions and to finetune the ML model using a second plurality of target molecule descriptions.

Clause 20. The apparatus of clause 19, wherein to pretrain the ML model using the first plurality of general molecule descriptions, the at least one processor is configured to pretrain the ML model using molecule descriptions from a public library of molecule descriptions.

Clause 21. The apparatus of any of clauses 19 to 20, wherein to finetune the ML model using the second plurality of target molecule descriptions, the at least one processor is configured to finetune the ML model using molecule descriptions of drug candidates.

Clause 22. The apparatus of clause 21, wherein to finetune the ML model, the at least one processor is configured to finetune the ML model using low-rank adaptation (LoRA).

Clause 23. The apparatus of clause 22, wherein to finetune the ML model using LoRA, the at least one processor is configured to introduce additional trainable rank-decomposition matrices into each layer of the neural network, while keeping the original pre-trained weights frozen.

Clause 24. The apparatus of any of clauses 14 to 23, wherein to generate the chemical structures using the trained ML model, the at least one processor is configured to: set an output string to an initial value; calculate, for each token in the vocabulary of the line notation, a probability of that token being the next token in the output string; select, from the vocabulary of the line notation, a next token to be appended to the output string, using a random selection according to the calculated probabilities; append the next token to the output string; and repeat the calculating, selecting, and appending steps until a maximum output string size is reached or an end token is selected from the vocabulary.

Clause 25. The apparatus of clause 24, wherein to set the output string to an initial value, the at least one processor is configured to set the output string to an empty string.

Clause 26. The apparatus of clause 24, wherein to set the output string to the initial value, the at least one processor is configured to set the output string to the value of an input prompt.

Clause 27. An apparatus comprising a memory, a transceiver, and a processor communicatively coupled to the memory and the transceiver, the memory, the transceiver, and the processor configured to perform a method according to any of clauses 1 to 13.

Clause 28. An apparatus comprising means for performing a method according to any of clauses 1 to 13.

Clause 29. A non-transitory computer-readable medium storing computer-executable instructions, the computer-executable comprising at least one instruction for causing a computer or processor to perform a method according to any of clauses 1 to 13.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those of skill in the art will appreciate that the various illustrative logical blocks, modules, circuits, and algorithm steps described in connection with the aspects disclosed herein may be implemented as electronic hardware, circuitry, computer software, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the disclosure.

The various illustrative logical blocks, modules, and circuits described in connection with the aspects disclosed herein may be implemented or performed with a general purpose processor, a DSP, an ASIC, an FPGA, or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods, sequences and/or algorithms described in connection with the aspects disclosed herein may be embodied directly in hardware, in a software module executed by a processor, or in a combination of the two. A software module may reside in random access memory (RAM), flash memory, read-only memory (ROM), erasable programmable ROM (EPROM), electrically erasable programmable ROM (EEPROM), registers, hard disk, a removable disk, a CD-ROM, or any other form of storage medium known in the art. An example storage medium is coupled to the processor such that the processor can read information from, and write information to, the storage medium. In the alternative, the storage medium may be integral to the processor. The processor and the storage medium may reside in an ASIC. The ASIC may reside in a user terminal (e.g., UE). In the alternative, the processor and the storage medium may reside as discrete components in a user terminal.

In one or more example aspects, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

While the foregoing disclosure shows illustrative aspects of the disclosure, it should be noted that various changes and modifications could be made herein without departing from the scope of the disclosure as defined by the embodiments and claims disclosed herein. The functions, steps and/or actions of the methods in accordance with the aspects of the disclosure described herein need not be performed in any particular order. Furthermore, although elements of the disclosure may be described or claimed in the singular, the plural is contemplated unless limitation to the singular is explicitly stated.

What is claimed is:

1. A method for generating chemical structure descriptions, the method comprising:
configuring a machine learning (ML) model trained on a line notation for describing chemical structures, the line notation having a vocabulary of tokens; and
generating, using the ML model, chemical structures described using the line notation for describing chemical structures.

2. The method of claim 1, wherein the ML model comprises an autoregressive large language model.

3. The method of claim 1, wherein configuring the ML model comprises:
training the ML model using a training set comprising molecules described using the line notation for describing chemical structures; or
receiving and installing a pre-trained ML model.

4. The method of claim 1, wherein the line notation for describing chemical structures is simplified molecular-input line-entry system (SMILES).

5. The method of claim 1, wherein training the ML model comprises pretraining the ML model using a first plurality of general molecule descriptions and finetuning the ML model using a second plurality of target molecule descriptions.

6. The method of claim 5, wherein pretraining the ML model using the first plurality of general molecule descriptions comprises pretraining the ML model using molecule descriptions from a public library of molecule descriptions.

7. The method of claim 5, wherein finetuning the ML model using the second plurality of target molecule descriptions comprises finetuning the ML model using molecule descriptions of drug candidates, finetuning the ML model using low-rank adaptation (LoRA), or both.

8. The method of claim 7, wherein the ML model comprises a neural network and wherein finetuning the ML model using LoRA comprises introducing additional trainable rank-decomposition matrices into each layer of the neural network, while keeping original pre-trained weights frozen.

9. The method of claim 1, wherein generating the chemical structures using the ML model comprises:
setting an output string to an initial value;
calculating, for each token in the vocabulary of the line notation, a probability of that token being the next token in the output string;
selecting, from the vocabulary of the line notation, a next token to be appended to the output string, using a random selection according to the calculated probabilities;
appending the next token to the output string; and
repeating the calculating, selecting, and appending steps until a maximum output string size is reached or an end token is selected from the vocabulary.

10. The method of claim 9, wherein setting the output string to the initial value comprises setting the output string to an empty string or to a value of an input prompt.

11. An apparatus, comprising:
a memory; and
at least one processor communicatively coupled to the memory, the at least one processor configured to:
configure a machine learning (ML) model trained on a line notation for describing chemical structures, the line notation having a vocabulary of tokens; and
generate, using the ML model, chemical structures described using the line notation for describing chemical structures.

12. The apparatus of claim 11, wherein the ML model comprises an autoregressive large language model.

13. The apparatus of claim 11, wherein to configure the ML model, the at least one processor is configured to train the ML model using a training set comprising molecules described using the line notation for describing chemical structures or to receive and install a pre-trained ML model.

14. The apparatus of claim 11, wherein the line notation for describing chemical structures is simplified molecular-input line-entry system (SMILES).

15. The apparatus of claim 11, wherein to train the ML model, the at least one processor configured to pretrain the ML model using a first plurality of general molecule descriptions and to finetune the ML model using a second plurality of target molecule descriptions.

16. The apparatus of claim 15, wherein to pretrain the ML model using the first plurality of general molecule descriptions, the at least one processor is configured to pretrain the ML model using molecule descriptions from a public library of molecule descriptions.

17. The apparatus of claim 15, wherein to finetune the ML model using the second plurality of target molecule descriptions, the at least one processor is configured to finetune the ML model using molecule descriptions of drug candidates, to finetune the ML model using low-rank adaptation (LoRA), or both.

18. The apparatus of claim 17, wherein the ML model comprises a neural network and wherein to finetune the ML model using LoRA, the at least one processor is configured to introduce additional trainable rank-decomposition matrices into each layer of the neural network, while keeping original pre-trained weights frozen.

19. The apparatus of claim 11, wherein to generate the chemical structures using the ML model, the at least one processor is configured to:
set an output string to an initial value;
calculate, for each token in the vocabulary of the line notation, a probability of that token being the next token in the output string;
select, from the vocabulary of the line notation, a next token to be appended to the output string, using a random selection according to the calculated probabilities;
append the next token to the output string; and
repeat the calculating, selecting, and appending steps until a maximum output string size is reached or an end token is selected from the vocabulary.

20. The apparatus of claim 19, wherein to set the output string to the initial value, the at least one processor is configured to set the output string to an empty string or to a value of an input prompt.
